(19) United States
(12) Patent Application Publication
(10) Pub. No.: US 2026/0272482 A1
(43) Pub. Date: Sep. 17, 2026

(54) ADAPTIVE PRESSURE-CYCLICAL ASPIRATION DEVICE AND METHODS

(71) Applicant: Von Vascular, Inc., Weston, FL (US)

(72) Inventors: Victor GAMEZ, Fort Lauderdale, FL (US); Manning J. HANSER, Weston, FL (US); Alfonso HERMIDA, Miramar, FL (US)

(73) Assignee: Von Vascular, Inc., Weston, FL (US)

(21) Appl. No.: 19/669,270

(22) Filed: May 6, 2026

Related U.S. Application Data
(63) Continuation of application No. 18/123,973, filed on Mar. 20, 2023, now Pat. No. 12,642,542.
(60) Provisional application No. 63/474,167, filed on Jul. 26, 2022.
(60) Provisional application No. 63/335,168, filed on Apr. 26, 2022.
(60) Provisional application No. 63/325,778, filed on Mar. 31, 2022.
(60) Provisional application No. 63/321,706, filed on Mar. 20, 2022.

(51) Int. Cl. A61B 17/22; A61M 1/00; A61B 17/00

(52) U.S. Cl. CPC A61B 17/22; A61M 1/74; A61B 2017/00022; A61B 2017/00199; A61B 2017/22079

(57) ABSTRACT

An aspiration system operable for relieving occlusions caused by clots and other lodged bodies in a vessel uses a cyclic algorithm which is adaptable to receipt of any known or developed aspiration catheter to treat acute ischemic occlusions by applied alternating catheter pressure within a defined range. The application of the algorithm is customizable by the specific physical characteristics of the catheter selected, making the system universally applicable to a significant range of catheters. A user interface may be provided conveniently within reach of the user enabling interactive input of intervening selections based on recommendations by a processor running the algorithm.

### CROSS-REFERENCE TO RELATED APPLICATIONS

This application is a continuation of U.S. Application No. 18/123,973, filed March 20, 2023, entitled “ADAPTIVE PRESSURE-CYCLICAL ASPIRATION DEVICE AND METHODS,” which claims the benefit of U.S. Provisional Application No. 63/321,706, filed March 20, 2022, entitled NOVEL ENHANCED ACUTE ISCHEMIC STROKE ASPIRATION ENERGY SOURCE,” U.S. Provisional Application No. 63/325,778 filed March 31, 2022, entitled “NOVEL ENHANCED ACUTE ISCHEMIC STROKE ASPIRATION SOURCE,” U.S. Provisional Application No. 63/335,168, filed April 26, 2022, entitled “NOVEL ENHANCED ASPIRATION SOURCE,” and U.S. Provisional Application No. 63/474,167, filed July 26, 2022, entitled “ALGORITHMS FOR NOVEL ENHANCED ASPIRATION SOURCE.”

### BACKGROUND OF THE INVENTION

The present disclosures relate, inter alia, to improved acute ischemic event therapies, and equipment and methods for implementing same to effectively and efficiently remove a clot occluding a vessel.

Conventional thrombotic extraction strategies for removing a clot occluding a blood vessel have routinely applied static aspiration. Attendant with such approach, plugging of a clot-extraction catheter had been heretofore encountered, thereby rendering continued use of the catheter essentially ineffective.

For example, when trying to aspirate a clot that is larger than the catheter, the clot often plugs the catheter according to a static aspiration approach. In particular, the clot is pulled partially into the catheter along a length thereof. The longer this length, the greater the friction there is present between the clot and the ID of the catheter. When the applied vacuum is alleviated or entirely discontinued, the clot expands slightly, but sufficiently so to push outward on the ID of the catheter, resulting in a risk that a catheter utilizing this approach will remain plugged and consequently rendered unable to effectively aspirate the clot if vacuum is restored.

By focusing upon static suction operating at or near full vacuum, for example, about -29.2 in Hg, without helpful or effective indication of tip activity, the prior art has heretofore been limited in lacking continuous feedback of conditions of the aspiration catheter. Although cyclic aspiration patterns have, to date, been suggested, the effectiveness and practice thereof has been severely limited.

Furthermore, because conventional aspiration systems generally still utilize static vacuum pumps, they have yet to enable reliably effective treatments for acute ischemic stroke, nor are they capable of universally interfacing with known and later developed aspiration catheters.

In this regard, some conventional aspiration systems employ dedicated catheter tubing sets sold and packaged separately. An additional drawback resides in the fact that the prior art clot extraction approaches are not compatible with neurovascular revascularization stent retrievers.

It would therefore be desirable to provide acute ischemic event therapies and optional wider functional applications which address and overcome these and other drawbacks of prior art approaches.

### SUMMARY OF THE INVENTION

In accordance with these and other objects of the invention, there is provided a thrombotic extraction system which provides the advantageous option of applying cyclic aspiration effective for fatiguing a clot occluding a vessel.

In particular, the invention relates to a system directed to recyclable pumps able to interface with known and later developed catheters, making them universally smart catheter systems, with touchscreen, WiFi, and/or Bluetooth controls for thrombus aspiration and treatment.

Briefly stated, energy sources with touchscreen enabled control of changes in cyclic forces drive AI-based algorithms guided by human medical intervention to support universal aspiration catheter interfacing for acute stroke therapy.

There are disclosed, inter alia, processes and methodologies regarding use of a recyclable pump constructed of sustainable materials for the purpose of aspirating thrombi, among other bodies, within blood vessels, particularly advantageously, those vessels within the human brain, and those associated with PV and/or STEMI, etc.

According to embodiments, universally interfaced cyclic algorithms drive use of any known or developed aspiration catheters to treat acute ischemic stroke by providing a novel enhanced energy source.

An aspiration system according to the invention comprises a pressure controllable pump system (conveniently in a form of an integrated pump device), including a peristaltic pump driven by a stepper motor. A sensor is advantageously included and is used to measure the fluid pressure in the catheter. This value serves to provide feedback to control logic which decides if the pump should generate positive or negative pressure to extract the clot. A combination of positive and negative pressures effectively forces the clot to reliably degrade and pass through the catheter.

In the case that the clot happens to plug the catheter, the algorithm instructs the pump to switch to a purely negative pressure mode (uniform vacuum) for a given time. If the pressure sensor indicates that the plugged or corked condition has changed, then the logic reverts back to a cyclical pressure mode for extracting the clot, sending corresponding instructions to the peristaltic pump.

If the measured pressure remains below a specified value, the system assumes that the clot has fully corked the catheter, and a message screen directs the user to remove the catheter while the pump is in constant aspiration mode (negative pressure) for example, full vacuum as employed in conventional systems.

A pressure sensor provided according to embodiment of the invention constantly monitors the pressure in the catheter and controls the pump accordingly in accordance with a selected algorithm suited to the aspiration catheter being used and which is advantageously implemented by processor control to stay above a lower pressure limit (Plower) and below an upper limit (Pupper).

In a particularly advantageous mode of operation, in which cycles of alternating pressure are applied, the clot is pulled towards or into the catheter under higher vacuum and released under lowered vacuum. This cycle advantageously repeats several times per second. The cyclic strain on the clot causes it to break under the applied repetitive alternating stress. When a broken off portion of the clot is smaller than the ID of the catheter, the catheter is operated so as to aspirate the clot under free-flow conditions. In accordance with an embodiment of the invention, if the vessel is still occluded, the catheter is advanced to the face of the clot again which causes the pressure in the catheter to be reduced indicating at least partial occlusion thereof, and the cyclic aspiration cycle is resumed.

In accordance with a particularly advantageous embodiment, when the device is activated, the pump aspirates blood out of the catheter and the pressure is monitored. If the pressure reaches the lower limit of the "smart range," the pump reverses and infuses blood into the catheter to raise the pressure. When the rising pressure reaches the upper limit of the "smart range," the pump reverses and aspirates again.

For purposes herein, the term "Smart Range" refers to a selected and controlled lower and upper range of vacuum values (the terms "PLower" and "PUpper," respectively). Similarly, the term "Smart Mode" identifies an option setting which serves to cyclically approach the lower and upper range without exceeding either, controlled by a selected algorithm suited to a particular catheter being used.

It has been found that higher frequencies break up a clot faster and more effectively than other approaches. The frequency of the cycling is dependent on how fast the pump can change the pressure. Parameters controlling frequency relate to software, hardware and system considerations.

For example, software parameters that affect frequency include predetermined selected upper and lower pressure limits (PUpper and PLower), speed and acceleration rates of the peristaltic pump rotor and sampling frequency of the pressure sensor.

Hardware parameters that come into play include compliance of tubing from sensor to catheter (stiffness and length), compliance of tubing from sensor to pump (stiffness and length), stiffness of pump tubing and volume per revolution of the pump rotor (tube ID and rotor diameter).

System parameters which also serve to determine cycle frequency include compliance of the catheter being used, compliance and characteristics of the particular clot being aspirated and amount of any air being present between the clot and the pump rotor.

The parameter thought to have a particular impact on cyclic frequency, and thus performance of the inventive approach, is the amount of air in the system. Consequently, it is highly preferable that the system be fully primed before use and stay primed during subsequent operation. For example, a loose RHV (Rotating Hemostasis Valve) may allow air to enter the system under vacuum and adversely impact the effectiveness of the applied cyclic determining algorithm.

In accordance with a particularly advantageous embodiment, the pump can self-prime at the start of a procedure. According to this approach, once the user tracks the aspiration catheter, bring the tip near the location of the clot, the pump can be connected and primed. The connection between pump and catheter (or catheter RHV) can potentially introduce an air bubble into the tubing. But the distance from the bubble to the pump rotor is fixed and therefore the volume is known. The pump can be operated to aspirate this fixed volume (ideally including an added safety factor) to prime itself.

A thusly primed pump will operate at a relatively high cycling frequency. Any air introduced into the system will reduce substantially such frequency. The system can optionally and advantageously monitor the cycling frequency, and provide feedback to the user at any time reduced frequency is detected, indicating the likelihood that the system needs to again be primed.

The control system determining the performance state of the pump optionally instructs the pump to operate as a purely static aspiration device, or as a smart device that uses the pressure sensor to determine how to best remove a clot.

In accordance with this optional embodiment, there are two operational modes. These include employment of an Adaptive Pulsative Algorithm (APA Mode) and a Static Mode in which the pump aspirates at uniformly maintained vacuum with no feedback from the provided pressure sensor.

Since the system optionally uses a positive displacement pump, such as for example a peristaltic type pump, the amount of blood pumped is proportional to the rotation(s) of the pump rotor and pressure. The pump can calculate and monitor the volume of blood pumped throughout the thrombotic extraction procedure. This volume can optionally be displayed to the user.

The blood volume can be compared to a settable limit by the processor which can optionally stop or slow the pump if this limit is reached. The user can then be alerted of this condition and may be presented with options for proceeding.

According to a particularly advantageous feature of the invention, the system can detect a plugged catheter by monitoring the pressure as the pump aspirates. If the pressure remains above the lower limit PLower of the smart range, the pump is free-flowing and the user has not made enough contact with the clot to cause the catheter to experience reduced flow. If the pressure drops to the lower limit of the "smart range" and remains so, the catheter is determined as being plugged. The system can advantageously present this information to the user to give feedback on the presence of the clot.

In summary, it is believed that no one has heretofore combined positive and negative cycling pressure that can support longer and more flexible aspiration catheters with improved touchscreens to control the cycling patterns for all catheters on the market and being improved all of the time.

By providing an optimized (as opposed to maximized) vacuum pressure/aspiration flow rate/thrombus removal force, the pressure system for the first time places all under interactive operator control.

A clot aspiration pump system according to embodiment of the present invention advantageously operates as a vacuum/aspiration pump with a compact footprint, operable for allowing a physician to extract thrombus through their choice of an on label aspiration thrombectomy catheter or stent retriever catheters adapted to use of aspiration.

Additionally, a system according to embodiment of the invention is advantageously provided in a form which is environmentally friendly. As envisioned, the housing is optionally and advantageously sourced from recycled materials and additionally, a clean take-apart process can optionally liberate and separate individual components for specialty material recycling and refurbishing.

A particularly advantageous feature of a clot aspiration pump is the system's compatibility with third-party thrombectomy catheters and/or stent retrievers. The pump system is advantageously configured as a sterile, single-use, battery-powered unit, and being compatible with aspiration indicated catheters.

The unit advantageously comprises in combination three components in one integrated device, including the components of tubing, a collection canister (reservoir), and an aspiration pump. A pump system according to embodiment of the current invention is advantageously agnostic to a specific catheter, controlled by software developed based upon features of current commercially available aspiration catheters.

A user-friendly touch screen is advantageously provided, which allows for the operator/physician to be engaged with the status of the pump and choose to activate the pump in either in adaptive mode (APA) or continuous aspiration (STATIC) mode.

The APA mode allows a pressure sensor output to adjust the operation of the pump, whereas the continuous aspiration mode (STATIC) is consistent with the actions of single-mode commercially available static aspiration pumps.

In accordance with a particularly advantageous embodiment, a pump system is integrated into a single unit which comprises a sterile single-use pump, standard nylon tubing, a sealed/disposable collection canister, integrated software, and a touchscreen display. Also, optionally, a battery pack and audio capabilities implemented by functional elements including, for example at least one speaker and associated audio components.

The touchscreen advantageously displays relevant conditions, such as for example, the battery level, status, operational mode, duration/time, pressure reading, and a graphical display of pressure, and allows the operator to select the appropriate catheter ID/length from a drop-down menu.

An advantageous embodiment according to the invention is intended to operate in a continuous aspiration and an adaptive mode. The continuous aspiration mode (STATIC), and the adaptive mode (APA) allows pressure sensor output to adjust the operation of the pump, allowing non-continuous aspiration.

In STATIC mode, the current device operates in a manner essentially equivalent to conventional static vacuum aspiration pumps.

A system-provided catheter inline pressure sensor provides the measured information to a system microprocessor, which in turn instructs the pump to vary the amount and type of aspiration according to predetermined parameters. This feature advantageously mitigates patient blood loss, and provides a facilitated means for disrupting the morphology of the thrombus, resulting in effective thrombus removal and a decrease in catheter blockage.

According to embodiments, there is disclosed a truly recyclable pump system for ischemic stroke treatment, programmable and adjustable via touchscreen interfaces.

While several embodiments of the present disclosure are described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles "a" and "an," as used herein in the specification and in the claims, unless clearly indicated to the contrary, should be understood to mean "at least one." The phrase "and/or," as used herein in the specification and in the claims, should be understood to mean "either or both" of the elements so conjoined, i.e., elements that are conjunctively present in some cases and disjunctively present in other cases. Other elements may optionally be present other than the elements specifically identified by the "and/or" clause, whether related or unrelated to those elements specifically identified, unless clearly indicated to the contrary.

Reference throughout this specification to "one embodiment" or "an embodiment" means that a particular feature, structure, or characteristic described in connection with the embodiment is included in at least one embodiment. Thus, appearances of the phrases "in one embodiment" or "in an embodiment" in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to "one embodiment," "an embodiment," or similar language means that a particular feature, structure, or characteristic described in connection with the embodiment is included in at least one embodiment of the present invention.

Thus, appearances of the phrases "in one embodiment," "in an embodiment," and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method.

Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method.

Additionally, the order in which a particular method occurs may or may not strictly adhere to the order of the corresponding steps or methodologies shown.

Unless otherwise indicated, all numbers expressing quantities of ingredients, properties such as molecular weight, reaction conditions, and so forth used in the specification and claims are to be understood as being modified in all instances by the term "about." Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms "a," "an," "the" and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., "such as") provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term "consisting of” excludes any element, step, or ingredient not specified in the claims. The transition term "consisting essentially of" limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term "machine-readable medium" should be taken to include a single medium or multiple media (e.g., a centralized or distributed database, and/or associated caches and servers) that store the one or more sets of instructions. The term "machine-readable medium" shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, "cloud" storage location, or a combination thereof. In certain embodiments, a device of the invention includes a tangible, non-transitory computer readable medium for memory. Exemplary devices for use as memory include semiconductor memory devices, (e. g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

Various preferred embodiments are described herein with references to the drawings in which merely illustrative views are offered for consideration, whereby: Corresponding reference characters indicate corresponding components throughout the several views of the drawings. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

The above, and other objects, features and advantages of the present invention will become apparent from the following description read in conjunction with the accompanying drawings.

Features, aspects, and advantages are described below with reference to the drawings, which are intended to illustrate, but not to limit, the invention. In the drawings, like reference characters denote corresponding features consistently throughout similar embodiments.

Corresponding reference characters indicate corresponding components throughout the several views of the drawing. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention.

Also, common but well-understood elements that are useful or necessary in a commercially feasible embodiment are often not depicted in order to facilitate a less obstructed view of these various embodiments of the present invention.

### BRIEF DESCRIPTION OF THE DRAWINGS

FIG. 1A is a CAD model perspective view depicting an example of a pump system in accordance with an embodiment of the invention;

FIG. 1B is a top plan view of the pump system embodiment of FIG. 1A;

FIG. 2 depicts a schematic representation of core components of a system according to an embodiment of the present invention;

FIG. 3 is a perspective view of a CAD model depicting an example according to an embodiment of the invention;

FIG. 4 is a schematic diagram which depicts an aspiration pump system according to an embodiment of the invention;

FIG. 5 is a simplified flow chart visually depicting how an APA mode algorithm according to an embodiment of the invention is implemented;

FIG. 6 is a graphical representation of cyclic aspiration performed over time when in APA mode according to an embodiment of the invention;

FIGS. 7A and 7B combined are a simplified operational flow chart depicting a basic operation of a pump system according to an embodiment of the invention;

FIGS. 8A and 8B combined are a flow chart depicting the methodologies of the algorithm main loop according to an embodiment of the invention;

FIGS. 9A and 9B combined are a flow chart depicting an aspirate methodology loop of an algorithm according to an embodiment of the invention;

FIGS. 10A, 10B, and 10C combined are a flow chart depicting an infuse methodology loop of an algorithm according to an embodiment of the invention;

FIG. 11 is graphical representation of pump flow stages associated with a basis clot extraction process with minimal issues;

FIG. 12 is graphical representation of pump flow stages associated with a clot extraction process during partial occlusion;

FIG. 13 is a graphical representation of pump flow stages associated with a clot extraction process terminating in a fully occluded catheter;

FIG. 14 is detailed graphical representation of pump flow stages associated with a start of the EXTRACT state;

FIG. 15 is a detailed graphical representation associated with an end of the EXTRACT state;

FIG. 16 is an annotated flow chart outlining a recycling and reprocessing model according to an exemplary embodiment of the invention;

FIG. 17A is a side perspective view of a system according to an embodiment of the invention which includes an integral clot catcher;

FIG. 17B is a top perspective view of the embodiment of FIG. 17A; and

FIG. 18 is an explanatory drawing outlining two cases of sidewall issues, including kink and bifurcation.

### DETAILED DESCRIPTION OF THE PREFERRED EMBODIMENT

Terms used herein as defined according to the intended definitions in the context of the present disclosure are now set forth herein.

Advantageously, there are two modes of operation. The SMART Mode which uses the Adaptive Pulsative Algorithm when in the APA Mode, and STATIC Mode in which the pump aspirates at essentially constant vacuum with no feedback from the pressure sensor.

The term "SMART Mode" is used herein to refer to the mode in which the pump system operates as a "smart device" controlled by an Adaptive Pulsative Algorithm ("APA mode") unique to a selected aspiration catheter, and constant aspiration in which the control system operates the aspiration as a purely static aspiration device ("STATIC Mode").

The terms "PUpper" and "PLower' refer respectively to defined upper and lower pressure limits used during APA mode.

Regarding use of the term "infusion," it will be understood that while often used in medical terms to describe an introduction of fluids, for example drugs, into the bloodstream, as used herein the term refers specifically to a reversal of aspiration resulting in an increase of catheter pressure.

Before referring to the drawings, a discussion of the various advantages of embodiments of the invention are presented.

Because of the approach of the invention, a direct connection of the catheter to the pump results in making possible shorter tubing lengths compared with convention aspiration pump systems. Additionally, for example, some systems employ catheter tubing sets sold and packaged separately. In accordance with embodiment of the present invention, the pump system can be supplied with its own connected, sterile tubing.

Embodiment of the present invention optionally provides audiovisual feedback during a thrombotic extraction procedure (i.e. w/ video screen). Audio and visually displayed information within the physician's reach and line of sight advantageously allows rapid response by the physician/operator in selecting operational changes as needed. A speaker can optionally verbally indicate to the operator conditions at the tip of the catheter, and can also give prompts.

Automatic shutoff according to an embodiment of the invention when a clot is cleared and also in "Free Flow" results in less patient blood loss. The blood displacement in Free Flow is significantly less than in conventional aspiration systems.

Embodiments of the present invention are capable of producing pressure cycles for effective removal of different types of clot using different catheter inner diameters. Positive and negative (vacuum) pressure cycle frequencies are adjustable for improved clot absorption/extraction (APA mode). Adjustable to catheter sizes and styles, proprietary algorithms for APA/cyclical suction for all catheters are possible.

Accelerated clot removal setting can increase the intensity of the frequencies when tougher clots are encountered.

Embodiments of algorithms of the invention are customizable to be formulated for soft clots, organized clots, atherosclerotic clots, and dense/fibrous clots.

An embodiment according to the invention is optionally compatible with neurovascular revascularization Stent Retrievers (in SR mode).

Embodiments according to the invention are capable of producing a vacuum pressure ofat least Full Vacuum (e.g., -29.7mmHg), and capable of being set to a "steady(static) vacuum" mode (in addition to the cyclic (APA) modalities).

In accordance with an optional embodiment, the pump system is at least partially recyclable.

According to an advantageous embodiment, the pump system has a built-in clot catcher in the canister. Optionally integrated into the actual pump or included inside the tubing.

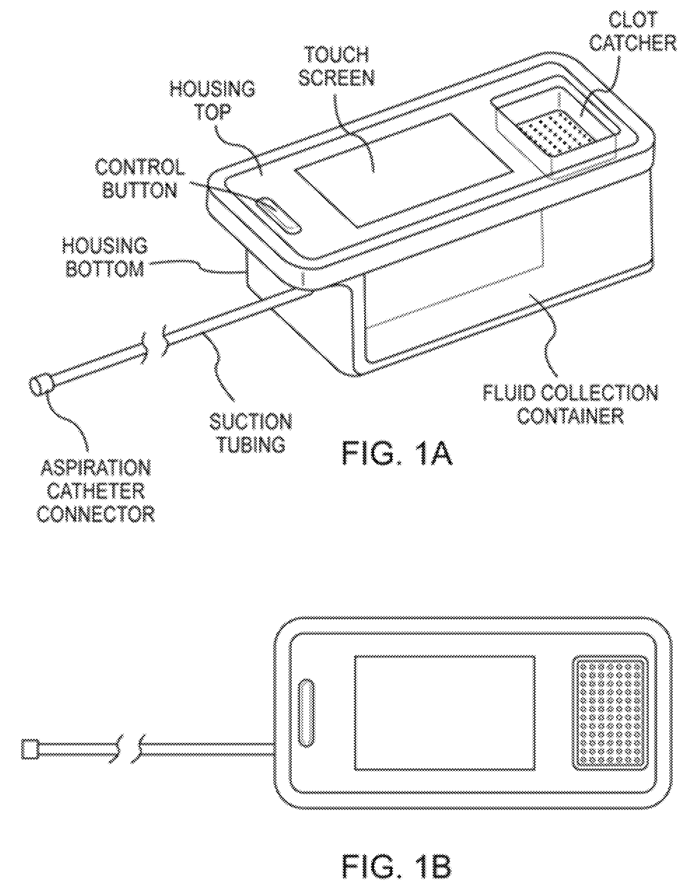

Turning now to FIGS. 1A and 1B, a CAD model of an example of a pump system according to the invention is shown in perspective view (FIG. 1A) and a top plan view (FIG. 1B).

As depicted, the exterior of the example of the pump device in FIGS. 1A and 1B comprises a suction tubing carrying a standard luer connector for hermetic mating with a conventionally available aspiration catheter, a housing, a touchscreen, a control button, a collection container, and a clot catcher for trapping a removed clot for examination. The internal components will be described in greater detail with reference to further figures.

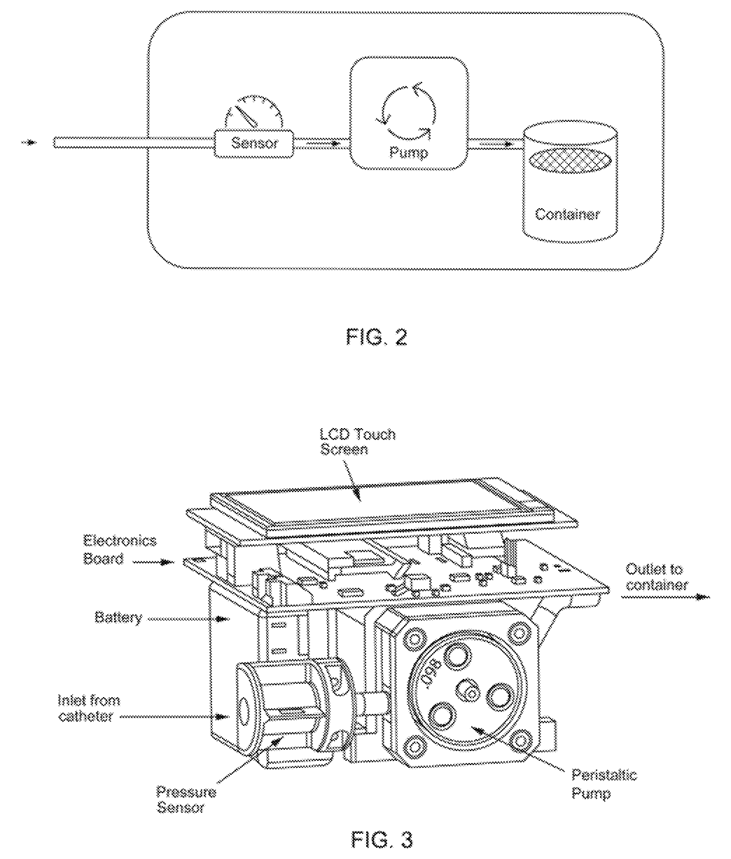

Referring to FIG. 2, a schematic representation of a core of a pump system according to the invention is shown, and which allows optional flexibility of a "Smart Pump" using selected algorithms designed for given aspiration catheters. As shown, a sensor is provided inline with the inlet tubing leading to a pump, advantageously provided by example as a peristaltic pump of a peristaltic pump driven by a stepper motor which includes an outlet connected with a container for collecting a removed clot or portions thereof. The sensor is used to measure the fluid pressure in the catheter, and this value is used by provided control logic to decide if the pump should generate positive or negative pressure to extract the clot, as will be described in greater detail.

According to a particularly advantageous embodiment, the stepper motor operates with micro stepping set at 32. The stepper motor takes 200 steps/revolution (360°/1.8°) when setup as full steps. With a setting of 32 microsteps/step the motor can perform 200 steps x 32 microsteps/step, or a total of 6400 steps/revolution. Commands to the stepper motor advantageously occur every 20ms.

As mentioned, the algorithm that controls the stepper motor uses the pressure sensor as the feedback device. Within the algorithm there are a set of parameters that define the upper and lower pressure limits and the speeds for clockwise (CW) and counterclockwise (CCW) rotation of the stepper motor.

An example of a suitable stepper motor is a standard NEMAl 7 stepper motor. The motor uses, for example, a DRV8825 stepper motor driver-based circuit that requires commands to be in the form of numbers of steps and direction (CW or CCW). In the example, the provided circuit board sets the microsteps to 32, making one revolution equal to 6400 microsteps.

The algorithm in its most basic form defaults to ASPIRATION mode. In this regard, when the control button is pressed, the algorithm sends a command to perform a large amount of steps (in this case, microsteps) at a constant rotational speed (microsteps/sec).

If the pressure sensor output is greater than the PLower pressure limit, the algorithm will continue commanding the motor to keep moving at the same rotational speed.

The algorithm runs on various loops. One of them will continue to send a new command to move the same amount of steps and at the same speed as long as the pressure sensor output indicates the system is in a FREE FLOW state (no interaction with the clot).

If a pressure drop occurs due to a clot covering the catheter tip and the pressure sensor output is below the PLower limit, the algorithm will command the motor to immediately change the direction of the motor. This starts an increase in pressure within the catheter to push the clot enough to loosen it from within the catheter.

This process repeats itself until the clot is aspirated fully and the pressure sensor output returns to the Free Flow pressure value or the occlusion cannot be resolved.

The algorithm operates by setting operational states, with the main states being ASPIRATE, INFUSE and STATIC PLUG.

ASPIRATE and INFUSE are the most demanding and require a fast response both from the sensor and the stepper motor/pump. In order to make sure that the response is not slowed down by unnecessary command checks, the main loop consistently checks at 20ms intervals the position and velocity of the stepper motor. Each command sent to the pump takes longer than 20ms to complete. Therefore, advantageously, every time an interrupt is triggered within the microprocessor, the status of the pump and the pressure sensor are checked and a new command is sent with updated values.

This guarantees that the pump will not remain idle at any point during the switch between ASPIRATE and INFUSE states. In addition, the system can quickly respond to changes in pressure due to changes in the interaction between the clot and the catheter.

At the beginning of the extraction process, the user presses the control button to start. The algorithm starts with aspiration at a constant rate.

If at any given time the tip of the catheter is in contact with the clot, the pressure sensor reading will drop. There are two events that create this pressure drop.

If the clot does not fully cover the catheter opening, the pressure will drop to some level. If this level is above the pressure lower limit (PLower) the pump will continue to aspirate at a consistent rate.

If the clot fully covers the catheter, the pressure will continue to drop. One of the following cases will apply.

If the clot is fully aspirated due to the increased suction, the pressure level will return to initial levels and the pump will remain in aspiration mode until the user stops the pump to terminate the process.

If the clot remains fully covering the catheter, the pressure level will drop until the PLower limit is reached. At this point, the algorithm commands the stepper motor to change direction and now increase the pressure.

At this point the pump is rotating CW in order to increase the pressure within the catheter in order to dislodge the clot slightly. Once the pressure reaches the PUpper limit, the algorithm instantly commands the stepper motor to aspirate. This process of changing stepper motor direction will repeat itself until one of two things occurs.

The clot is fully aspirated. The stepper will remain in aspiration mode until stopped by user.

After a predetermined amount of time of increasing and decreasing pressure cycles the algorithm will switch to a static aspiration mode (STATIC mode). In this mode, the pressure drops to vacuum and allows (if possible) the clot to be aspirated. This mode also has a predetermined time limit. If the clot is not aspirated within this time, a screen is displayed indicating that the catheter needs to be removed. The pump will maintain the vacuum state until the user presses the control button.

As will be discussed further, a combination of positive and negative pressures force the clot to degrade and pass through the catheter. In the case that the clot plugs the catheter, an applied algorithm will switch to a purely negative static pressure mode for a given time. If the pressure sensor indicates that the plugged or corked condition has changed, then the logic reverts back to extracting the clot. If the pressure remains below a specified value, the logic of the system assumes that the clot has fully corked the catheter, and a message screen advantageously directs the user to remove the catheter while the pump is in constant aspiration mode (negative pressure).

FIG. 3 depicts a perspective view of a CAD model showing an example of an embodiment of an aspiration pump system according to an embodiment of the invention. As shown, the illustrated pump system includes pressure sensor, inlet from the catheter, a peristaltic pump, a battery power supply, an electronics board for microprocessor control of the pump, an LCD touch screen and an outlet to a collection container.

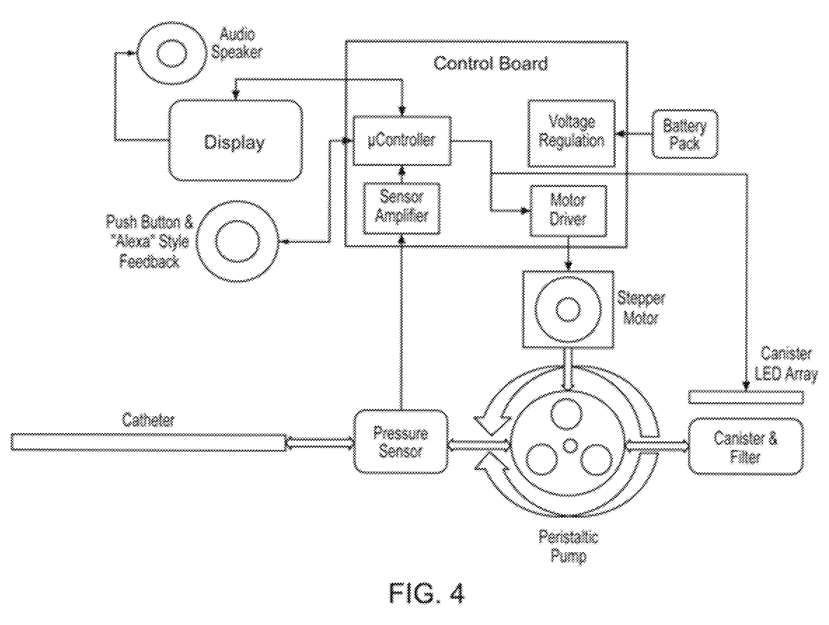

A schematic diagram detailing the elements of an advantageous embodiment of a pump system of the invention is depicted in FIG. 4. The depicted pump diagram shows the embodiment as comprising a display, a control board, a button and a battery pack. The control board advantageously includes a micro processor controller, a sensor amplifier, a voltage regulator and a motor drive.

A catheter leads to the pressure sensor which is connected with a peristaltic pump driven by a stepper motor, and an outlet of which is connected to canister and clot collection filter.

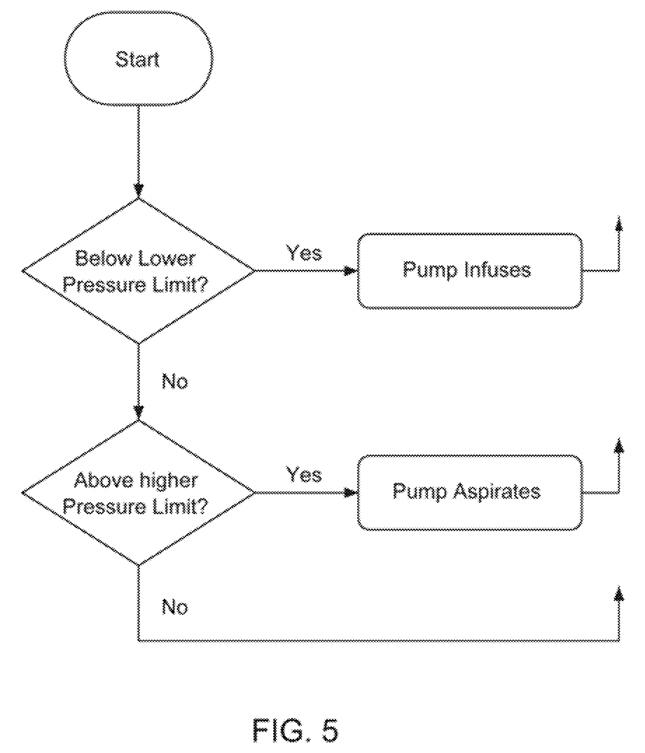

Referring to FIG. 5, a simplified flow chart visually depicting how an APA mode algorithm according to an embodiment of the invention is implemented. After the system is started, the sensor reports whether the pressure in the catheter is below a lower pressure limit (Plower). If yes, the pump is instructed to infuse.

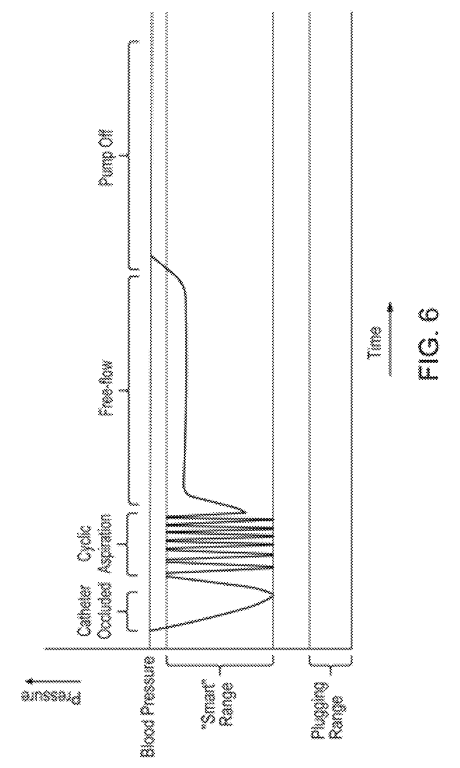

FIG. 6 graphically represents pressure variances over time when cyclic aspiration is performed in APA mode according to an embodiment of the invention. Cyclic aspiration is applied, maintaining oscillating pressure conditions varying between PLower and PUpper.

For purposes of disclosure, it is noted that there are five modes (flow states) in which the pump according to embodiment of the invention operates within. These include a READY mode, a FREE FLOW mode, an EXTRACT mode, a PLUG mode and a REMOVAL mode. Each is addressed below.

In READY mode, the pump has been primed and is ready to operate. The pump begins operation once the start/stop control button (see FIGS. 1A and 1B, and 4) is pressed.

When in FREE FLOW mode, the pump is in aspiration mode (negative pressure). The measured fluid pressure is below a defined upper value (Pupper) and above a lower defined value (PLower), and therefore no occlusion or partial occlusion is assumed.

When in EXTRACT mode when the pressure value drops below a defined value, it indicates that a partial or full occlusion of the catheter has occurred. The algorithm constantly generates positive and negative pressures based on the pressure levels measured. The amount of time that the pump is in the EXTRACT state is stored and compared to a defined limit. If this limit is exceeded the pump state switches to PLUG, indicating a blockage of the catheter.

Consequently a PLUG mode is initiated when the EXTRACT state exceeds a defined amount of time. The algorithm switches to a PLUG state, indicating that the pressure is lower than the defined level (PLower). The pump continues to create negative pressure for an additional amount of time. If the pressure increases above the defined Plower value, the pump returns to the EXTRACT state. If not, a message indicates that the catheter is corked, and it is recommended that the catheter be removed. The pump state switches to REMOVAL mode.

In REMOVAL mode, the pump maintains a constant negative pressure to allow the removal of the catheter with the clot attached or inside of it. Once the catheter is removed, the user must press the start/stop button to switch the pump back to READY.

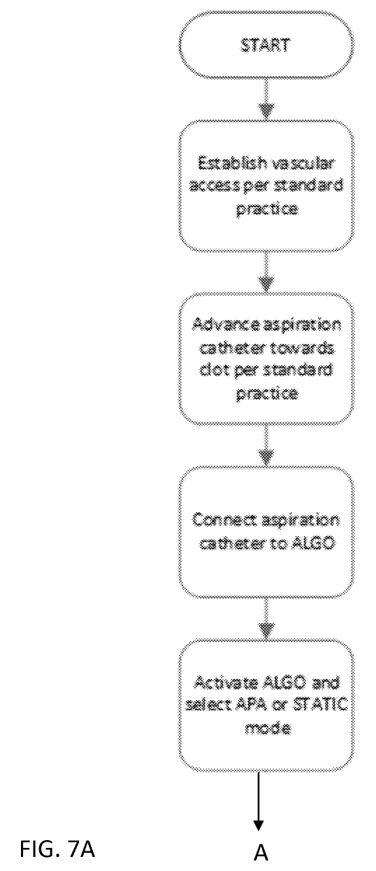

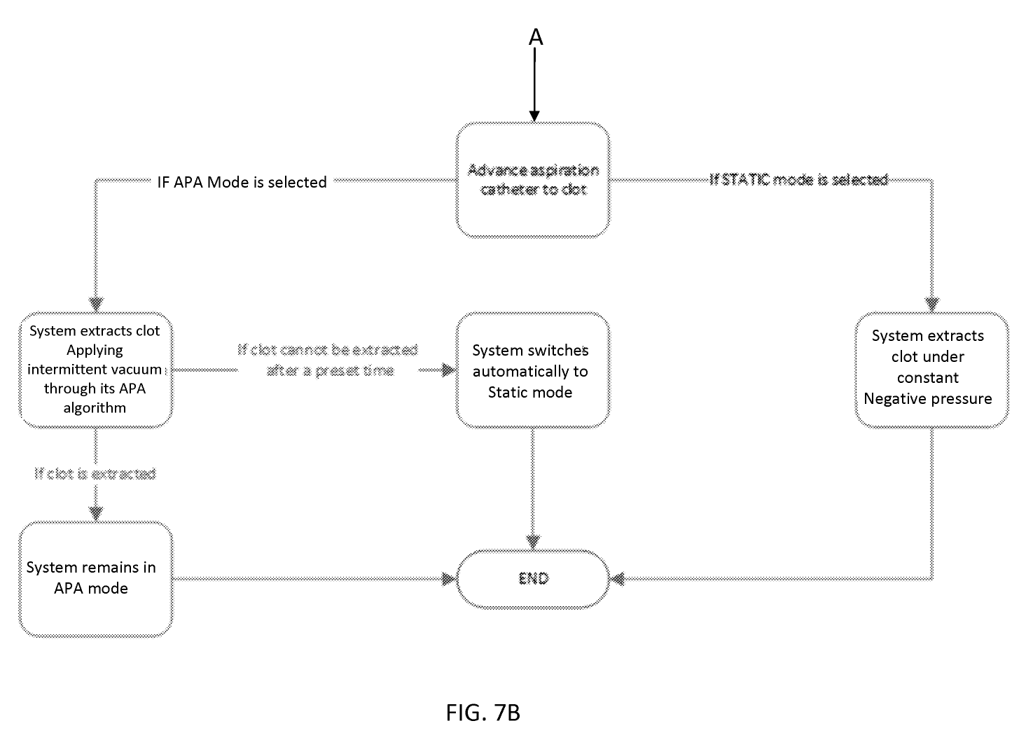

FIGS. 7A and 7B are a flow chart depicting process methodologies from START to END for both STATIC and APA modes.\Reference to "operational state" is used to describe the pump behavior along a main loop depending on the current flow state discussed above, as controlled by a selected pump algorithm.

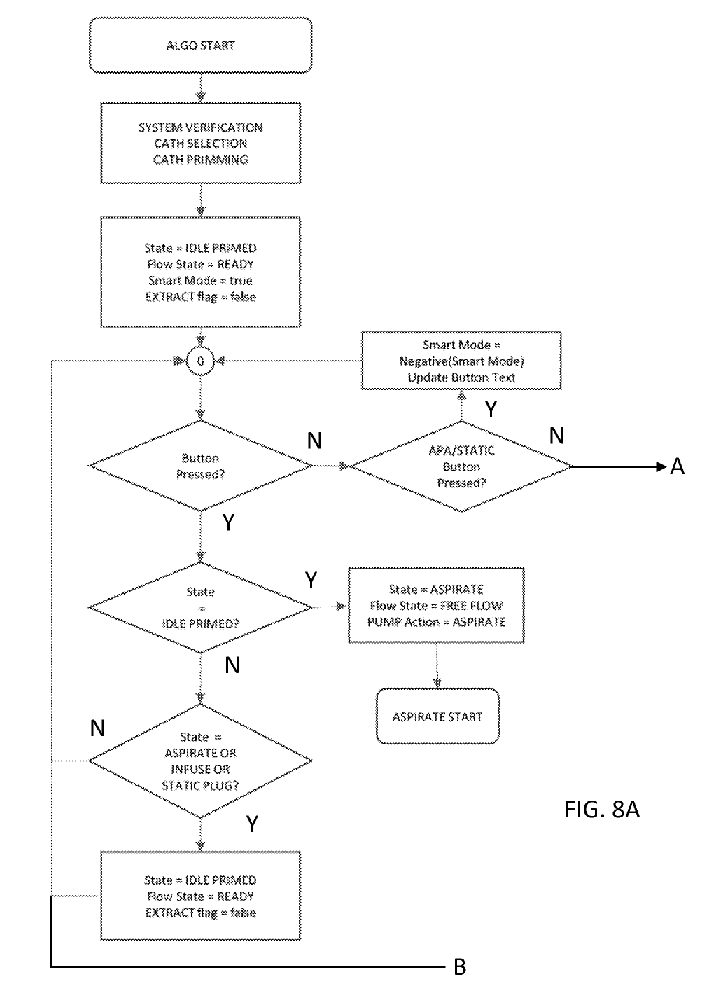

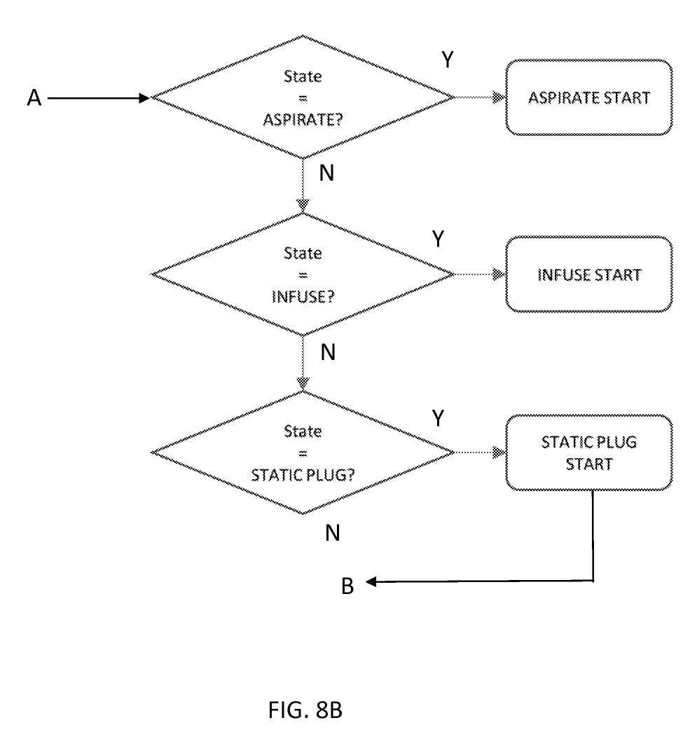

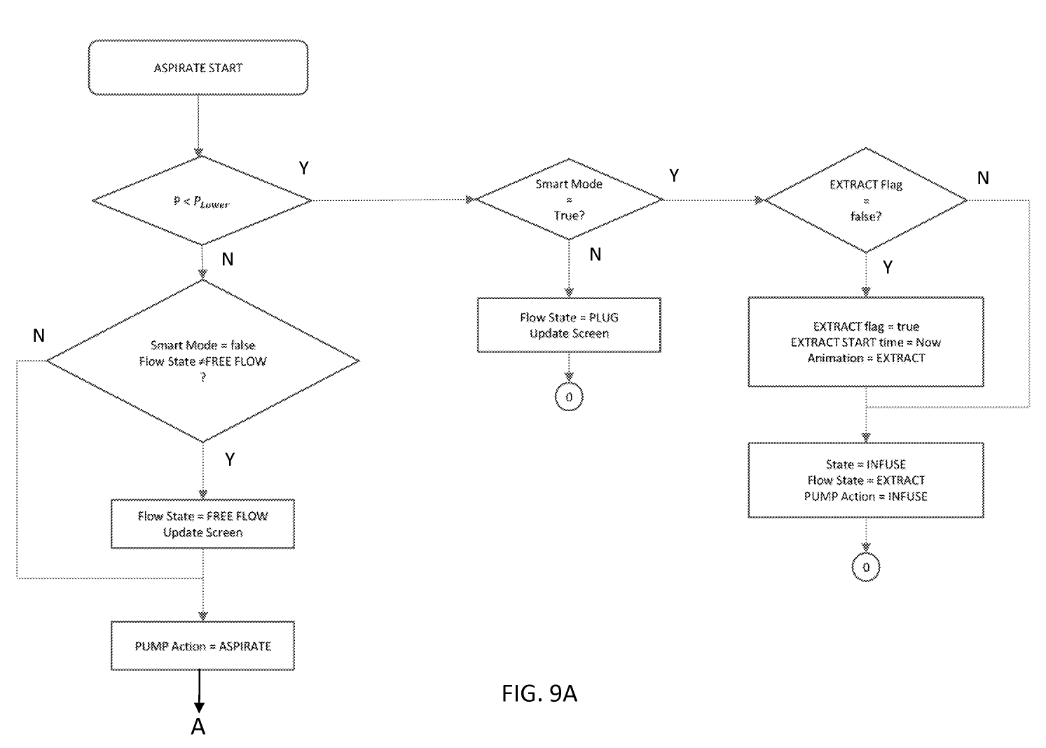

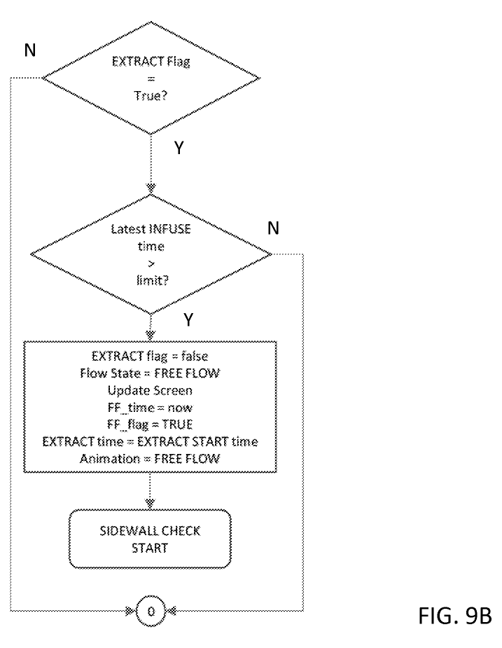

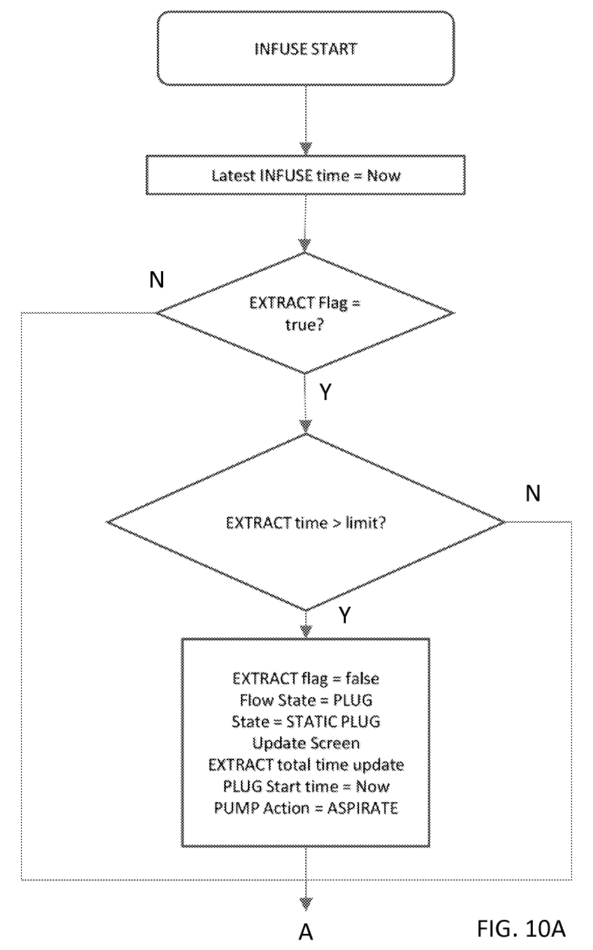

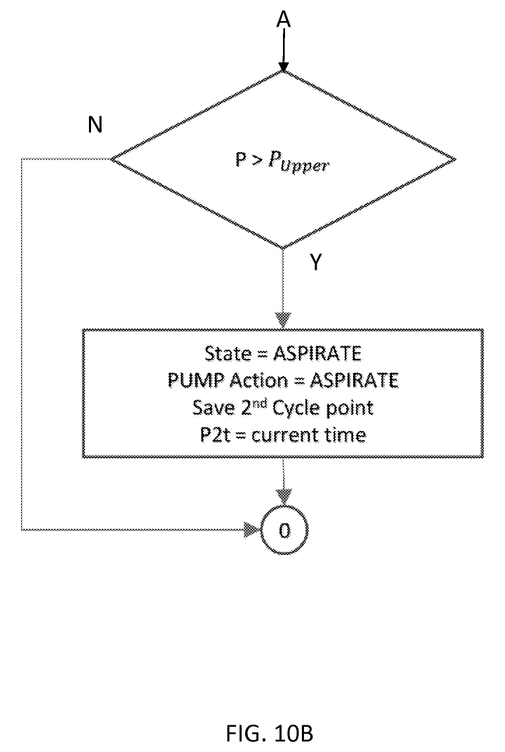

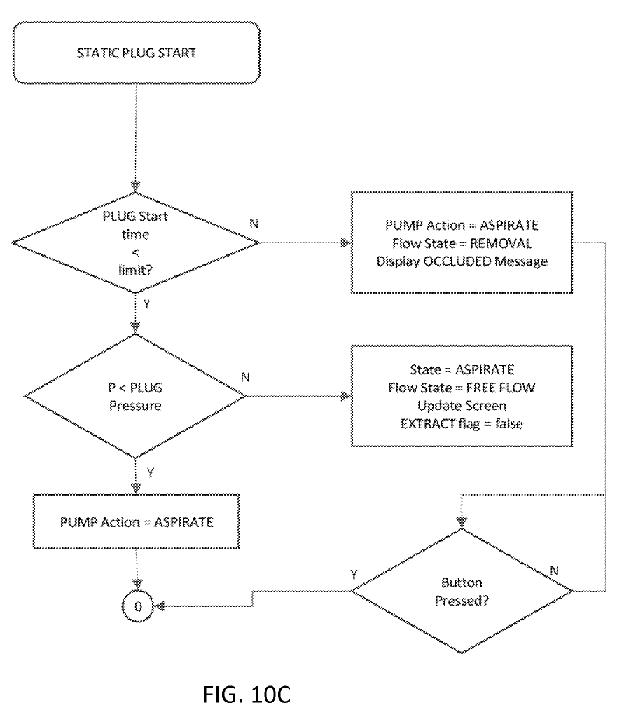

Referring now to FIGS. 8A and 8B, a main loop is depicted in the illustrated flow chart. The flow chart begins in a start block and terminates in any of a start of ASPIRATE, INFUSE or STATIC PLUG. Each of these selections are depicted as flow charts in FIGS. 9A-10B.

Turning now to FIGS. 9A-9B, the depicted process begins with a start of ASPIRATE. The pump generates negative pressure creating a suction effect. If the pump is operating with the Smart Mode Off (STATIC mode) the aspiration will continue at a constant rate independent of the pressure sensor. If the Smart Mode is On (APA mode) the algorithm will determine how much to aspirate based on the pressure sensor value.

Referring to FIGS. 10A-10C, in INFUSE state, the pump generates a positive pressure on the fluid. This operational state only occurs during the EXTRACT state in APA mode. The algorithm will determine how much to INFUSE based on the pressure sensor value.

Also in FIGS. 10A-10C, a STATIC PLUG control state is shown. During the STATIC PLUG state, the pump maintains a negative pressure for a specified time. This allows the clot to be affected by the pressure and break to thereby allow partial fluid flow or simply allow the clot to pass through the catheter. When the STATIC PLUG state has been active for less than a specified time, the Flow State is set to PLUG. If no changes occur to the clot, based on the pressure sensor values, then the Flow State switches to REMOVAL. During the PLUG Flow State, a change in the pressure can trigger a return back to the EXTRACT state. Once the Flow State has been set to REMOVAL, the algorithm maintains a static negative pressure until the start/stop button has been pressed.

The selected algorithm uses various pressure level values to control the pump operation. These include blood pressure (BP), upper positive pressure limit allowed by the pump ≤ BP (PUpper), lower negative pressure limit to trigger clot removal, and Atmospheric pressure (ATM).

The pump controller according to an embodiment of the invention keeps track of the total amount of time the unit has been ON and the time the current EXTRACT state has been active. These and other additional values can be stored with the processor for later retrieval.

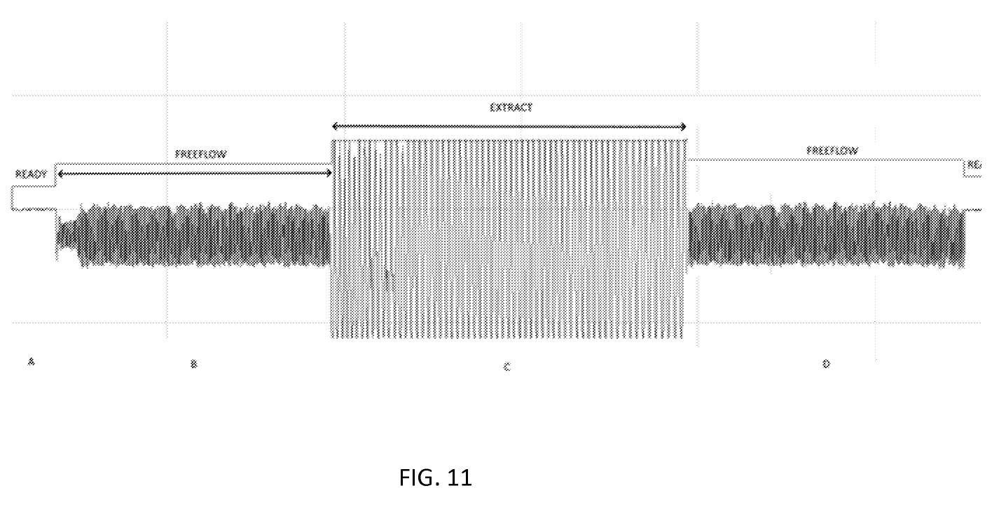

Turning now to FIG. 11, a graphical representation displays the flow seen in a basic extraction where the clot was successfully removed with minimal issues. The process shown in FIG. 11 is detailed below.

Initially, the pump is in READY state (section A). Once the start/stop control) button is pressed, the pump aspirates (generated negative pressure) but this pressure drop is not low enough to indicate partial or full occlusion of the catheter, and therefore it is determined to be in the FREE FLOW state (section B). As soon as the catheter makes contact with the clot, a partial or full occlusion occurs and the pressure drops. This activates the EXTRACT state (section C).

During the EXTRACT state, the algorithm determines if positive or negative pressure is required to force the clot to degrade and go through the catheter. Once this occurs, the pressure increases and the pump returns to the FREE FLOW state (section D).

Once the start/stop button is again pressed, the pump stops and the pressure of the fluid returns to a stable value.

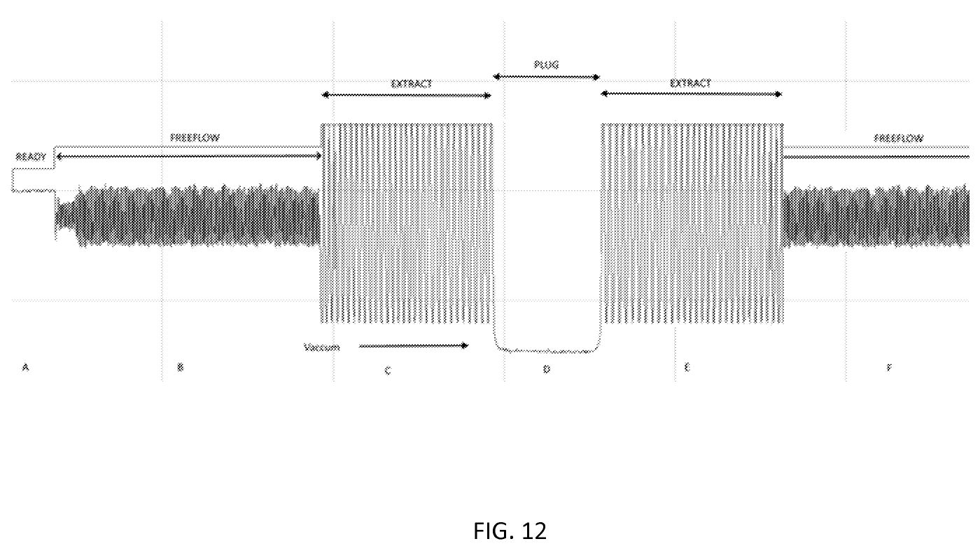

Referring to FIG. 12, a graph shown therein is directed to control of pump flow during a partial catheter occlusion. FIG. 12 displays a case in which the EXTRACT state is not able to remove the clot completely during a defined amount of time. The pump goes into the STATIC PLUG state and keeps a negative pressure, which can be seen when the pressure line drops towards a vacuum. If the occlusion can be removed at least partially, the pump changes to the EXTRACT state. This process is allowed to repeat until time limits are exceeded or used stops the process. The process shown in FIG. 12 is detailed below:

Initially the pump is in READY state (section A). Once the start/stop button is pressed, the pump aspirates (generates negative pressure). But this pressure drop is not low enough to indicate partial or full occlusion of the catheter, and therefore it is determined to be in the FREE FLOW state (section B).

As soon as the catheter makes contact with the clot, a partial or full occlusion occurs and the pressure drops. This activates the EXTRACT state (section C).

During the EXTRACT state, should the algorithm exceed a limit amount of time to remove the clot, the pump changes to the STATIC PLUG state and maintains a negative pressure. This can be seen when the pressure line drops towards vacuum (section D).

If the occlusion is partially removed, the pump switches back to the EXTRACT state and restarts the process (section E).

Once the clot is removed, the pump returns to the FREE FLOW state (section F), and eventually to the READY state once the start/stop button is pressed (not shown in FIG. 2).

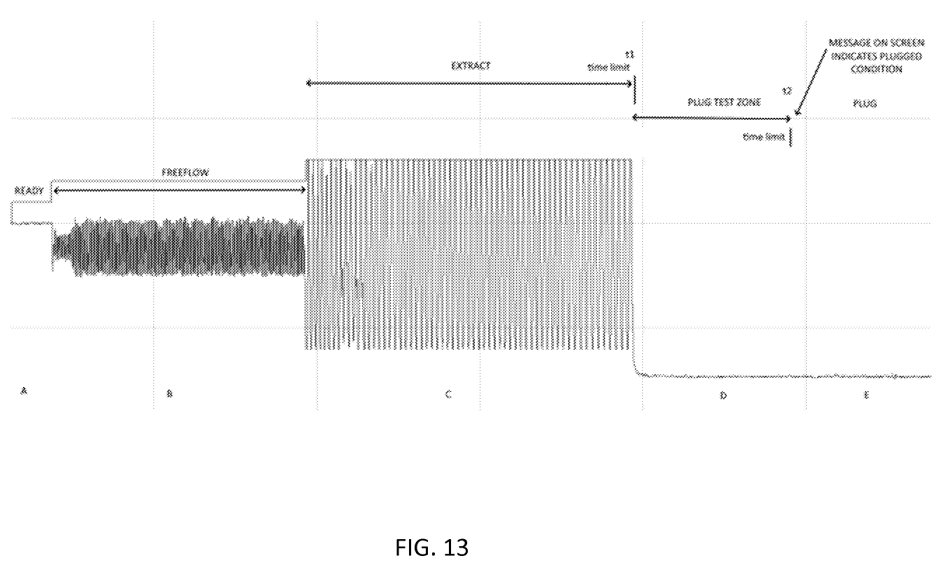

Turning now to FIG. 13, the depicted graph shows pump flow during full occlusion. In the case that the EXTRACT state exceeds the defined time limit, the pump switches to the STATIC PLUG state. During this state, the pump aspirates, and keeps a negative pressure creating a vacuum. This state also has a time limit. The purpose is to simply force a constant negative pressure on the clot and extract the clot. This state can be switched back to the EXTRACT state if the pressure increases. This period is considered a "test" period. Once the time limit is exceeded the algorithm stops trying to remove the clot, and simply keeps the negative pressure while the user removes the catheter carefully along with the clot. Once this is done, the user presses the start/stop button to return the pump to the READY state. The process shown in FIG. 13 is detailed below.

Initially, the pump is in READY state (section A). Once the start/stop button is pressed, the pump aspirates (generated negative pressure) but this pressure drop is not low enough to indicate partial or full occlusion of the catheter therefore is determined to be in the FREE FLOW state (section B).

As soon as the catheter makes contact with the clot, a partial or full occlusion occurs and the pressure drops. This activates the EXTRACT state (section C). During the EXTRACT state, the algorithm has exceeded the limit amount of time (t1) to remove the clot. The pump changes to the STATIC PLUG state and maintains a negative pressure. This can be seen when the pressure line drops towards vacuum (section D).

The STATIC PLUG state also has a time limit (t2), which in FIG. 13 is shown to have been exceeded. This forces the pump to switch to terminate the algorithm and display a help screen for the user (section E).

Once the user removes the catheter with the clot, the start/stop button should be pressed to set the pump in READY state, in case an additional pass is required.

In order for the employed algorithm to switch from FREE FLOW state to the EXTRACT state, the pressure drops to a value defined by PLower. This drop triggers the change of state and the algorithm, based on the current pressure value, commands the pump to aspirate (generate a negative pressure) or infuse (generate a positive pressure).

The EXTRACT state starting time occurs when the first infuse command is made by the algorithm. The EXTRACT state has a time limit to prevent the algorithm from operating in a "forever" clot extraction mode.

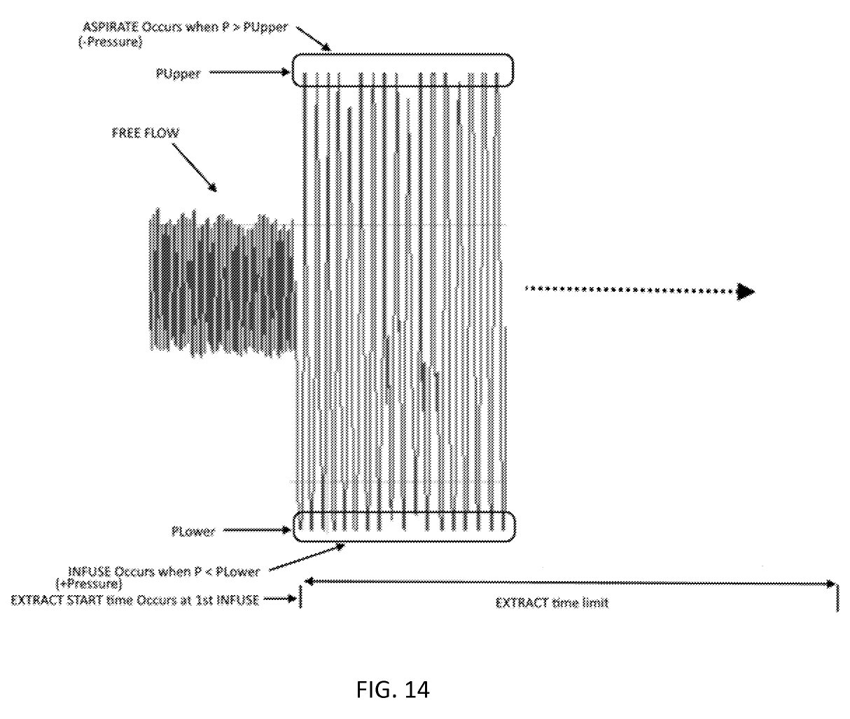

FIG. 14 presents the various pressure levels and time limits defined in the algorithm.

In order to determine when the EXTRACT state is complete and the FREE FLOW state should be active, the algorithm looks at the timing in the infuse (+Pressure) commands.

In the previous section (6.0) the beginning of the EXTRACT state starts when the pressure drops to PLower and the timestamp for the start of the EXTRACT section occurs when the Infuse (+Pressure) command is generated by the algorithm. During the EXTRACT state, the algorithm will determine when the aspirate (-Pressure) and infuse (+Pressure) commands are required.

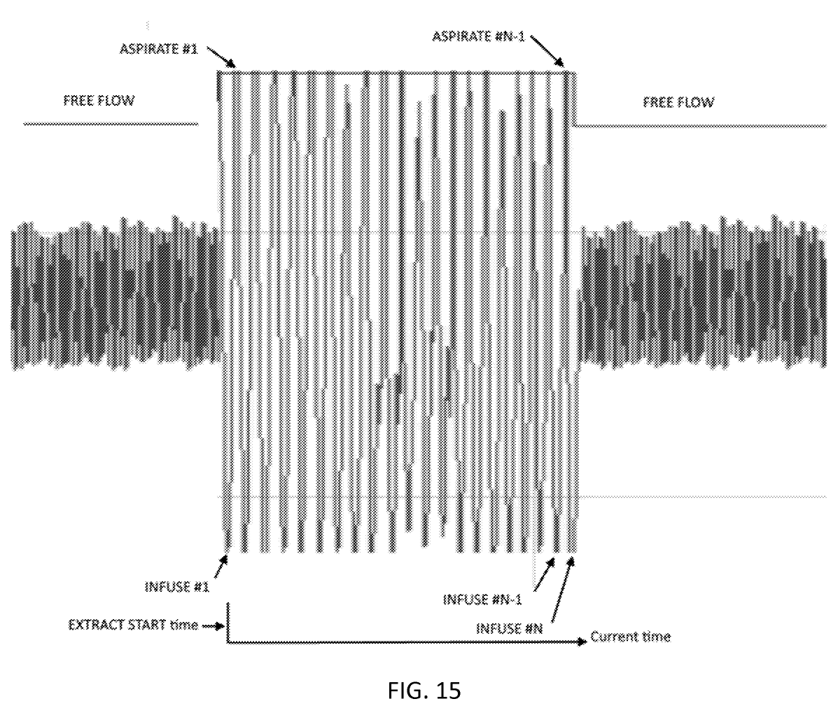

FIG. 15 displays a detailed depiction at the end of the EXTRACT state when the aspirate and infuse commands are executed. In order for the infuse command to be executed, the pressure has to drop to PLower. If the pressure does not drop, this is an indication that the occlusion is minimal or none. The algorithm will remain aspirating. If the time between the current time and last time the infuse command was executed exceeds a time limit, then the algorithm sets the state to be FREE FLOW. In FREE FLOW the pump is continuously generating negative pressure. The user has to determine if there are additional clots or if all have been removed.

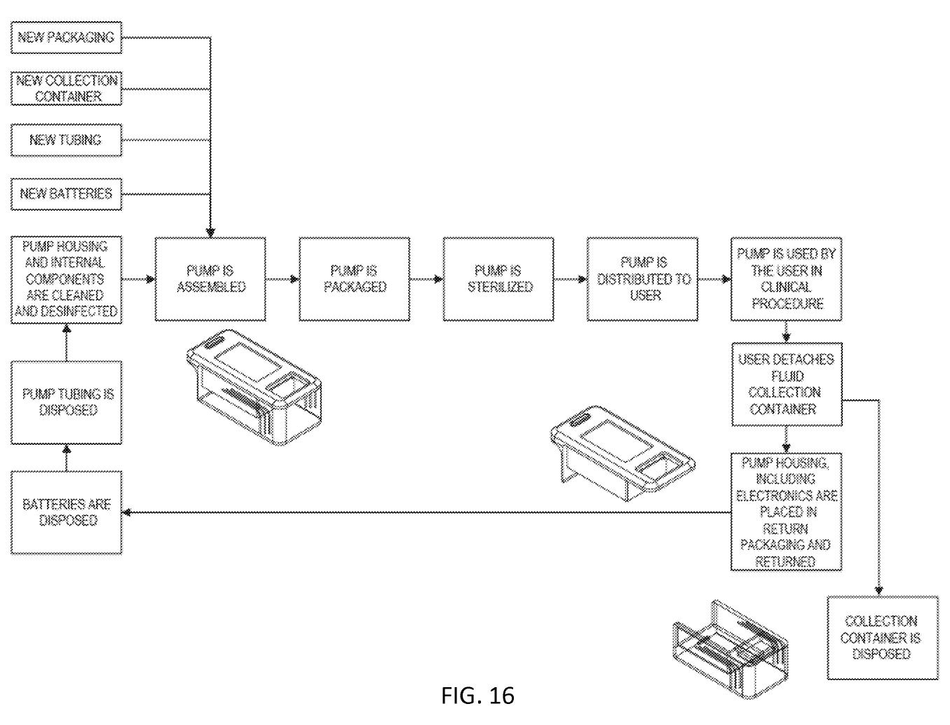

FIG. 16 is an annotated flow chart outlining a recycling and reprocessing model according to an exemplary embodiment of the invention, setting forth an example of recycling methodologies according to one possible example of the invention.

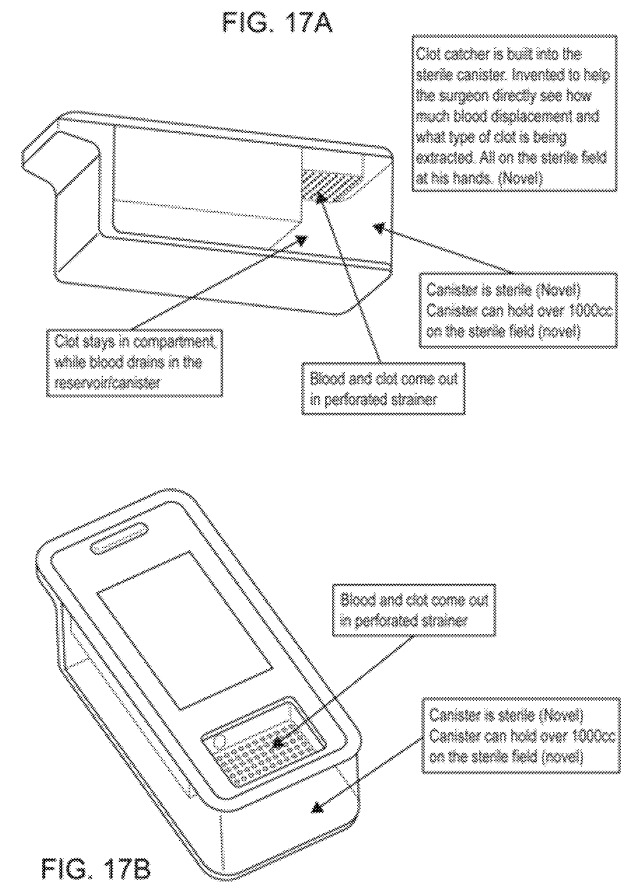

FIGS. 17A and 17B illustrates in two views, a version of the system According to a particularly advantageous embodiment, clot capture device (Clot Catcher), fully integrated with the pump blood collection container, that allows the capture and immediate removal of the aspired clot without exiting the sterile field. The Clot Catcher lid (see FIG. 17B) can be removed for collection of the extracted clot for subsequent examination and analysis. The clot catcher bottom is perforated as a sieve to allow the passage of blood while retaining the extracted clot.

The pump can also be operated with the cover of the clot catcher completely removed for even faster and easier clot removal by the user. All other conventional systems have their clot retrieval devices in line with the suction path. This requires the user to stop the pump to retrieve the clot. This means that c of the aspiration process to retrieve the clot. This invention beneficially allows the user to retrieve the extracted clot without stopping the aspiration process.

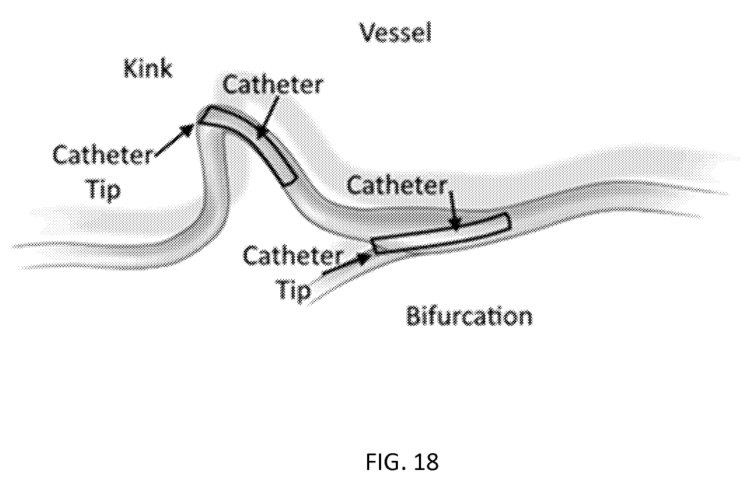

As depicted in FIG. 18, during a clot extraction procedure, a selected catheter is introduced within a vessel while aspirating. If the tip of the catheter is in contact with the wall of the vessel there are cases (sidewall) where the wall may cover the catheter tip as depicted in FIG. 18. As shown in the illustration, one possibility is an occurrence due to the catheter in contact with the vessel wall at a kink, and a second possibility is when the tip is located at a bifurcation.

During any of these conditions, the pump pressure sensor immediately senses a drop in pressure (due to the momentary occlusion). Once the pressure drops below the lower pressure limit (PLower) this triggers the pump to start the extraction mode.

When the sidewall case occurs, the algorithm starts cycling between increasing and decreasing the pressure in the catheter. This has the effect of separating the catheter slightly from the vessel wall. At this point the algorithm senses a free flow condition (no occlusion due to a clot or to contact with a vessel wall). The state in the algorithm changes to FREE FLOW.

If the catheter due to the tortuosity of the vessel again is covered by the vessel wall this process of toggling between EXTRACTION and FREE FLOW modes will repeat itself many times.

At the algorithm level, the sidewall case consists of multiple toggles between the extracting and free flow modes. An EXTRACTING and then FREE FLOW state normally occurs when the aspiration system has removed the clot. This pair of states occurs closely together but do not necessarily repeat at short time intervals (as it occurs during a sidewall case).

The time interval between repeated EXTRACTING/FREE FLOW states is compared against a time limit. Short intervals indicate a possible sidewall case is in effect. If the number of these events reaches an initial limit, a warning message is displayed suggesting that the catheter should be moved slightly to adjust the catheter tip location, thus removing the sidewall scenario. If a sidewall count variable exceeds a second limit then the pump is automatically switched to a STATIC pressure mode and a message is displayed indicating that the pump has been placed in STATIC pressure mode and that the catheter should be carefully retrieved. The static pressure will remain until the catheter is removed and the user presses a button indicating the process is complete. At this point all sidewall variables are reset and the pump is set to READY mode. At this point a new extraction process can be started.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention.

What is claimed is:

1. A method of aspirating a thrombus from a body lumen defined by a vessel via a catheter coupled to an aspiration system, the aspiration system including a motor, a controller implemented in at least one of a memory or a processor, and a sensor, the method comprising:
selecting, via the controller, an aspiration algorithm that includes a forward speed, and a backward speed;
sending, from the controller, a first command to the motor to operate the motor in a first extraction mode during which the motor is cycled between the forward speed and the backward speed for a plurality of cycles to produce a thrombus removal force at a tip of the catheter;
receiving, from the sensor, a signal associated with the thrombus removal force;
determining a presence of a signal characteristic of the signal, the signal characteristic being associated with contact between a side wall of the vessel and the tip of the catheter; and
sending, from the controller and on a condition that the signal characteristic is present, a second command to the motor to operate the motor in a second extraction mode, the second extraction mode different from the first extraction mode.

2. The method of claim 1, wherein:
the signal is a cyclic signal; and
the signal characteristic is associated with a time period between successive cycles.

3. The method of claim 1, wherein:
the signal is a cyclic signal; and
the determining the presence of the signal characteristic includes comparing a number of cycles during which the signal characteristic is present with a cycle threshold.

4. The method of claim 1, further comprising:
producing a notification on the condition that the signal characteristic is present.

5. The method of claim 4, wherein:
the aspiration system includes a collection container fluidically coupled to the catheter, the collection container configured to receive an aspirated fluid from the body lumen;
the aspiration system includes a visual output device coupled to a cover of the collection container; and
the notification is a visual output produced by the visual output device.

6. The method of claim 5, wherein the visual output device is a display screen.

7. The method of claim 1, wherein the signal characteristic is a first signal characteristic, the method further comprising:
determining a presence of a second signal characteristic of the signal, the second signal characteristic being associated with plugging of the thrombus within the tip of the catheter; and
sending, from the controller and on a condition that the second signal characteristic is present, a third command to the motor to operate the motor in a third extraction mode, the third extraction mode different from the first extraction mode.

8. The method of claim 7, further comprising:
producing a notification to withdraw the catheter from the body lumen on the condition that the second signal characteristic is present.

9. The method of claim 1, wherein:
the aspiration system includes a pump configured to be fluidically coupled to the catheter; and
the motor is operably coupled to drive the pump such that when the motor is operated in the first extraction mode, a catheter pressure cycles between an upper pressure limit and a lower pressure limit.

10. The method of claim 9, wherein:
the sensor is a pressure sensor; and
the signal associated with the thrombus removal force is the catheter pressure.

11. An aspiration system configured to aspirate a thrombus from a body lumen defined by a vessel via a catheter, the aspiration system, comprising:
a motor;
a sensor; and
a controller including at least one of a memory or a processor, the controller configured to:
(1) receive a user input, the user input associated with the catheter to be coupled to the aspiration system, (2) select an aspiration algorithm that includes, a forward speed, and a backward speed, (3) send a first command to the motor to operate the motor in a first extraction mode during which the motor is cycled between the forward speed and the backward speed for a plurality of cycles to produce a thrombus removal force at a tip of the catheter, (4) receive a signal associated with the thrombus removal force from the sensor, (5) determine a presence of a signal characteristic of the signal, the signal characteristic being associated with contact between a side wall of the vessel and the tip of the catheter, and (6) send a second command, on a condition that the signal characteristic is present, to the motor to the motor to operate the motor in a second extraction mode, the second extraction mode different from the first extraction mode.

12. The aspiration system of claim 11, further comprising:
a collection container fluidically coupled to the catheter, the collection container configured to receive an aspirated fluid from the body lumen;
a visual output device coupled to a cover of the collection container; and
the controller is configured to produce a notification via the visual output device on the condition that the signal characteristic is present.

13. The aspiration system of claim 12, wherein the notification includes an instruction to move the catheter.

14. The aspiration system of claim 12, wherein the visual output device is a display screen.

15. The aspiration system of claim 14, wherein the user input is received in response to a prompt on the display screen.

16. The aspiration system of claim 11, wherein:
the signal is a cyclic signal; and
the signal characteristic is associated with a time period between successive cycles.

17. The aspiration system of claim 11, wherein:
the signal is a cyclic signal; and
the presence of the signal characteristic is determined by comparing a number of cycles during which the signal characteristic is present with a cycle threshold.

18. The aspiration system of claim 11, wherein:
the signal characteristic is a first signal characteristic; and
the controller is further configured to determine a presence of a second signal characteristic of the signal, the second signal characteristic being associated with plugging of the thrombus within the tip of the catheter, and send, on a condition that the second signal characteristic is present, a third command to the motor to operate the motor in a third extraction mode, the third extraction mode different from the first extraction mode.

19. The aspiration system of claim 11, further comprising:
a pump configured to be fluidically coupled to the catheter, the motor being operably coupled to drive the pump such that when the motor is operated in the first extraction mode, a catheter pressure cycles between an upper pressure limit and a lower pressure limit.

20. The aspiration system of claim 19, wherein:
the sensor is a pressure sensor; and
the signal associated with the thrombus removal force is the catheter pressure.
